SEQUENCE LISTING (15 sequences)

SEQ ID NO: 1 - protein, 17 aa, Artificial Sequence
  Synthetic peptide
LLQLTVWGIKQLQARIL

SEQ ID NO: 2 - protein, 17 aa, Artificial Sequence
  Synthetic peptide
LQARILAVERYLKDQQL

SEQ ID NO: 3 - protein, 19 aa, Artificial Sequence
  Synthetic peptide
YTSLIHSLIEESQNQQEKN

SEQ ID NO: 4 - protein, 14 aa, Artificial Sequence
  Synthetic peptide
TTWMEWDREINNYT

SEQ ID NO: 5 - protein, 18 aa, Artificial Sequence
  Synthetic peptide
LLQLTVWGIKQLQARILK

SEQ ID NO: 6 - protein, 17 aa, Artificial Sequence
  Synthetic peptide
ELQLTQWKIKQLLARIL

SEQ ID NO: 7 - protein, 18 aa, Artificial Sequence
  Synthetic peptide
ELQLTQWKIKQLLARILK

SEQ ID NO: 8 - protein, 14 aa, Artificial Sequence
  Synthetic peptide
LLQLTVWGIKQLQA

SEQ ID NO: 9 - protein, 20 aa, Artificial Sequence
  Synthetic peptide
YTSLIHSLIEESQNQQEKNK

SEQ ID NO: 10 - protein, 15 aa, Artificial Sequence
  Synthetic peptide
TTWMEWDREINNYTK

SEQ ID NO: 11 - protein, 10 aa, Artificial Sequence
  Synthetic peptide
TWMEWDREIK

SEQ ID NO: 12 - protein, 10 aa, Artificial Sequence
  Synthetic peptide
IWMEWDREIK

SEQ ID NO: 13 - protein, 51 aa, Artificial Sequence
  Synthetic peptide
RQLLSGIVQQQNNLLRAIEAQQHLLQLTVWGIKQLQARILAVERYLKDQQL

SEQ ID NO: 14 - protein, 56 aa, Artificial Sequence
  Synthetic peptide
SLEQIWNHTTWMEWDREINNYTSLIHSLIEESQNQQEKNEQELLELDKWASLWNWF

SEQ ID NO: 15 - protein, 9 aa, Artificial Sequence
  Synthetic peptide
TWMEWDREI

(19) United States
(12) Patent Application Publication
(10) Pub. No.: US 2015/0045291 A1
(43) Pub. Date: Feb. 12, 2015

(54) LIPOPEPTIDE CONJUGATES COMPRISING SPHINGOLIPID AND HIV GP41 DERIVED PEPTIDES

(71) Applicant: YEDA RESEARCH AND DEVELOPMENT CO., LTD., Rehovot (IL)

(72) Inventors: Yechiel Shai, Yahud (IL); Avraham Ashkenazi, Rehovot (IL)

(21) Appl. No.: 14/390,372

(22) PCT Filed: Apr. 3, 2013
(86) PCT No.: PCT/IL2013/050305

§ 371 Date: Oct. 2, 2014

Related U.S. Application Data
(60) Provisional application No. 61/619,956, filed on Apr. 4, 2012.

(51) Int. Cl. A61K 47/48; C07K 14/005

(52) U.S. Cl. CPC A61K 47/48046; C07K 14/005
USPC 514/3.8; 530/326; 530/327; 530/325; 530/328; 435/375

(57) ABSTRACT

The invention provides conjugates comprising a short isolated peptide coupled to a sphingolipid, the peptide comprising a sequence derived from the HIV-1 gp41. The sphingolipid-peptide conjugates are suitable for treatment of infections caused by human and non-human retroviruses, especially HIV.

### FIELD OF THE INVENTION

The present invention relates to lipopeptide conjugates comprising a sphingolipid moiety coupled to peptides derived from the HIV-1 gp41, to pharmaceutical compositions comprising same, and use thereof as inhibitors of human and non-human retroviral, especially HIV, transmission to uninfected cells.

### BACKGROUND OF THE INVENTION

The first step in the life cycle of enveloped viruses is entry into their host cells by membrane fusion. To mediate fusion, the viral envelope protein (ENV) needs to assist in overcoming the energy barriers to fusion imposed by its surrounding lipids. Emerging studies on human immunodeficiency virus (HIV) and other viruses such as Influenza, West Nile, and Murine coronavirus suggest membrane-ordered domains as a lipid platform for virus release. This results in viral envelopes with elevated levels of ordered lipid domains. Ordered lipid domains are assemblies in cell membranes that are enriched in cholesterol and sphingolipids. The formation of these domains and the partitioning of proteins and lipids into them are dynamic processes. Therefore, it is of great importance to identify lipids that can be incorporated into the fusion site during the dynamics of the membrane fusion events.

The HIV ENV is composed of two non-covalently associated subunits: the gp120 subunit enables binding of cell receptors and co-receptors, whereas the gp41 transmembrane subunit mediates the physical membrane fusion reaction. Binding of gp120 to CD4 and co-receptor involves conformational changes in both gp120 and gp41, resulting in gp41 pre-hairpin conformation. This conformation is sensitive to gp41-derived peptide fusion inhibitors that capture it in an intermediate state. As a result, the folding of the protein into the hairpin conformation is prevented, leading to inhibition of viral fusion. The hairpin conformation comprises a trimeric central coiled-coil that is created by three N-terminal heptad repeat (NHR) regions, into which three C-terminal heptad repeat (CHR) regions are packed in an anti-parallel manner (Colman et al. Nat. Rev. Mol. Cell. Biol. 4, 309-319, 2003; Chan et al. Cell 89, 263-273, 1997; Weissenhorn et al. Nature 387, 426-430, 1997). The structure is usually referred to as the “six-helix bundle” or “core” structure, which is required for complete membrane fusion. Similar bundles are created in other viral fusion proteins and in intracellular vesicle fusion by soluble N-ethylmaleimide-sensitive factor accessory protein receptor (SNARE) proteins, demonstrating a common mechanism in diverse systems (Sollner, T. H. Curr. Opin. Cell Biol. 16, 429-435, 2004).

The capability of different peptides derived from the CHR region or NHR regions of HIV gp41 to inhibit transmission of HIV to host cells was demonstrated both in in vitro assays and in in vivo clinical studies. For instance, DP178, a 36-mer peptide (also known as T20, enfuvirtide, and Fuzeon®), fragments, analogs and homologs thereof having anti-retroviral activity, have been disclosed in several publication including: U.S. Pat. Nos. 5,464,933; 5,656,480; 6,093,794; 6,133,418; 6,258,782; 6,333,395; 6,348568; 6,479,055; 6,750,008; 7,122,190, 7,273,614 and 7,456,251.

Fuzeon is the first and only clinically approved fusion inhibitor. It shows good antiviral potency and long-term safety in clinical use. Nevertheless, the virus exhibited emerging drug-resistant strains to treatment with Fuzeon, and the compound has a relatively low genetic barrier for resistance. Hence there is a pressing need to expand the knowledge on the mechanism of HIV-cell fusion that could give rise to future microbicides.

U.S. Pat. No. 6,747,126 provides chimeric peptides comprising a soluble trimeric coiled-coil and all or a portion of the N-peptide region of HIV gp41.

US Patent application No. 2008/0199483 relates to peptides selected from DP178, DP107 and related peptides and analogs thereof, exhibiting anti-viral and anti-fusogenic activity modified to provide greater stability and improved half-life in vivo. The modified peptides have a reactive group such as succinimidyl or maleimido which are capable of forming covalent bonds with one or more blood components, preferably a mobile blood component.

US Patent Application No. 2008/0096809 of one of the inventors of the present invention relates to diastereomeric peptides derived from DP178 and DP107 peptides, wherein at least two amino acid residues of the diastereomeric peptide are in the D-isomer configuration, the modified peptides display increased solubility.

US Patent Application No. 2012/0028887 of one of the inventors of the present invention provides lipophilic conjugates comprising an isolated peptide coupled to a hydrophobic moiety, the peptide comprising a sequence derived from the HIV-1 gp41 N-terminal heptad repeat domain, said peptide after conjugation to the hydrophobic moiety possesses antiviral activity higher than prior to conjugation. The lipophilic conjugates are suitable for treatment of infections caused by human and non-human retroviruses, especially HIV.

While the prior art peptides derived from the CHR region or NHR regions of HIV gp41 have been shown useful in inhibiting viral transmission to uninfected cells, each has significant shortcomings as a therapeutic. The cost of manufacturing peptides rises exponentially with their increasing length. Their potential immunogenicity increases with their length as well. Another drawback associated with synthetic peptides relates to the solubility and stability in aqueous-based pharmaceutically acceptable carriers, such as relating to the process of making an injectable solution formulation of an HIV fusion inhibitor peptide. For example, it is difficult to achieve an injectable aqueous solution containing a synthetic peptide having an amino acid sequence of DP178 in a concentration of more than 100 mg/ml without encountering problems of solubility (wherein the formulation resembles a gel, rather than a solution, or peptide precipitates out of solution over a predetermined time period) and stability (peptide being degraded over a predetermined period of time).

None of the background art discloses or suggests that sphingolipids may endow nanomolar antiviral activity to short HIV-1 gp41 peptides, having otherwise no or minimal antiviral activity. Further, none of the background art discloses or suggests that conjugates of sphingolipids and the short gp41 peptides may be active against Fuzeon-resistant HIV. Thus, there is a need for an effective retroviral fusion inhibitory peptide, especially HIV fusion inhibitor peptide. The present invention addresses these needs.

### SUMMARY OF THE INVENTION

The present invention provides lipopeptide conjugates comprising a sphingolipid moiety and a peptide derived from HIV-1 gp41, and pharmaceutical compositions comprising same effective as inhibitors of human and non-human retroviral cell fusion.

The present invention is based, in part, on the unexpected discovery that sphingolipids exclusively endowed nanomolar antiviral activity to short (e.g., up to 22 residues) HIV-1 gp41 peptides, having otherwise no or minimal antiviral activity. Further, conjugates of sphingolipids and the short gp41 peptides were remarkably active against Fuzeon-resistant and wild-type CXCR4 and CCR5 tropic strains. Without wishing to be bound by theory or mechanism of action, the antiviral activity of the sphingolipids-peptides conjugates of the invention originated from their strong binding and accumulation in cell-membrane compartments.

Thus, the present invention provides sphingolipid-peptide conjugates comprising an isolated peptide of no more than 22 amino acid residues derived from HIV gp41, coupled to a sphingolipid moiety. The present invention further provides pharmaceutical compositions comprising said sphingolipid-peptide conjugates useful for inhibiting human and non-human retroviral activity, including but not limited to, virus replication, transmission or infection of a cell. In some embodiments the peptide corresponds to a fragment of the transmembrane protein HIV-1 gp41 core structure. In a particular embodiment, the peptide is derived from (i.e. corresponds to) amino acids 542 to 592 or 618 to 673 of HIV-1_{HXB2} gp160. In another embodiment, the peptide corresponds to a fragment of the transmembrane protein HIV-1 gp41 N-terminal heptad repeat (NHR). In another embodiment, the peptide corresponds to a fragment of the transmembrane protein HIV-1 gp41 C-terminal heptad repeat (CHR).

According to one aspect, the present invention provides a sphingolipid-peptide conjugate comprising an isolated peptide of up to 22 amino acid residues coupled to a sphingolipid moiety, the peptide comprising the amino acid sequence selected from the group consisting of:

LLQLTVWGIKQLQARIL (SEQ ID NO:1);

LQARILAVERYLKDQQL (SEQ ID NO:2);

YTSLIHSLIEESQNQQEKN (SEQ ID NO:3); and

TTWMEWDREINNYT (SEQ ID NO: 4); or an analog, a derivative or fragment thereof.

According to exemplary embodiments, the sphingolipid moiety is selected from sphinganine and sphingosine. Said sphingolipid moiety, according to some embodiments of the invention, is saturated, unsaturated, monounsaturated, or polyunsaturated. According to some embodiments, said sphingolipid moiety is conjugated to the N-terminus or C-terminus of said isolated peptide. According to another embodiment, said sphingolipid moiety is conjugated to the N-terminus of said isolated peptide. According to other embodiments, said sphingolipid moiety is conjugated to the C-terminus of the isolated peptide.

According to certain embodiments of the invention, the isolated peptide comprises up to 22 amino acid residues. According to some embodiments, the peptide comprises up to 21 amino acid residues. According to some embodiments, the peptide comprises up to 20 amino acid residues. According to some other embodiments, the peptide comprises up to 19 amino acid residues. According to yet other embodiments, the peptide comprises up to 18 amino acid residues. According to further embodiments, the peptide comprises up to 17 amino acid residues.

According to some embodiments, the peptide further comprises 1-4 basic amino acid residues contiguous to at least one of the peptide's termini. According to some embodiments, the 1-4 basic amino acid residues are conjugated to the N-terminus of said isolated peptide. According to some embodiments, the 1-4 basic amino acid residues are conjugated to the C-terminus of said isolated peptide. In particular embodiments, the basic amino acid residue is selected from Lysine and Arginine residues. In exemplary embodiments, the basic amino acid residue is Lysine. In yet another exemplary embodiment, a Lysine residue is contiguous to the N-terminus said isolated peptide. According to certain exemplary embodiments, the isolated peptide has an amino acid sequence as set forth in any one of:

LLQLTVWGIKQLQARILK (SEQ ID NO:5);

YTSLIHSLIEESQNQQEKNK (SEQ ID NO:9); and

TTWMEWDREINNYTK (SEQ ID NO:10).

Each possibility represents a separate embodiment of the present invention. According to some embodiments, the isolated peptide comprises the amino acid sequence as set forth in LLQLTVWGIKQLQARIL (SEQ ID NO:1). According to another embodiment, the isolated peptide consists of the amino acid sequence as set forth in SEQ ID NO:1. According to yet another embodiments, the isolated peptide comprises or consists of the amino acid sequence as set forth in LLQLTVWGIKQLQARILK (SEQ ID NO:5). According to another embodiment, the sphingolipid-peptide conjugate comprises or consists of an analog, a derivative, or fragment of SEQ ID NO:1. According to exemplary embodiments, the analog or fragment has the amino acid sequence selected from the group consisting of:

ELQLTQWKIKQLLARIL (SEQ ID NO:6);

ELQLTQWKIKQLLARILK (SEQ ID NO:7); and

LLQLTVWGIKQLQA (SEQ ID NO:8). Each possibility represents a separate embodiment of the present invention.

According to another embodiment, the isolated peptide comprises the amino acid sequence as set forth in LQARILAVERYLKDQQL (SEQ ID NO:2). According to another embodiment, the isolated peptide consists of the amino acid sequence as set forth in SEQ ID NO:2. According to yet another embodiment, the isolated peptide comprises or consists of an analog, a derivative or fragment of SEQ ID NO:2.

According to some embodiments, the isolated peptide comprises the amino acid sequence as set forth in YTSLIHSLIEESQNQQEKN (SEQ ID NO:3). According to another embodiment, the isolated peptide consists of the amino acid sequence as set forth in SEQ ID NO:3. According to another embodiment, the isolated peptide comprises or consists an analog, a derivative or fragment of SEQ ID NO:3. According to another exemplary embodiment, the isolated peptide comprises the amino acid sequence as set forth in YTSLIHSLIEESQNQQEKNK (SEQ ID NO:9). According to another embodiment, the isolated peptide consists of the amino acid sequence as set forth in SEQ ID NO:9.

According to additional embodiments, the isolated peptide comprises the amino acid sequence as set forth in TTWMEWDREINNYT (SEQ ID NO:4). According to another embodiment, the isolated peptide comprises or consists of TTWMEWDREINNYTK (SEQ ID NO:10). According to an exemplary embodiment the isolated peptide consists of the amino acid sequence as set forth in SEQ ID NO: 10. According to another embodiment, the isolated peptide comprises or consists an analog, a derivative or fragment of SEQ ID NO:4. According to another exemplary embodiment, the fragment of SEQ ID NO:4 consists of the amino acid sequence as set forth in TWMEWDREIK (SEQ ID NO:11).

According to some embodiments of the invention, the isolated peptide is selected from all L-amino acid peptides and diastereomeric peptides. According to some embodiments the peptide comprises at least 90% L-amino acids. According to other embodiments the peptide comprises at least 95% L-amino acids. According to an exemplary embodiment the isolated peptide consists of the amino acid sequence as set forth in IwMewDREIK (SEQ ID NO: 12), wherein “w” denotes D-Trp and “e” denotes D-Glu.

According to another aspect, the present invention provides a pharmaceutical composition comprising as an active ingredient a sphingolipid-peptide conjugate of the present invention and a pharmaceutically acceptable carrier or diluent.

The pharmaceutical composition may be formulated for any route of administration including, but not limited to, intravenous, intramuscular, intraperitoneal, nasal, intralesional and topical.

In one embodiment, the pharmaceutical composition is for inhibiting infection of a cell by a virus. In another embodiment, said pharmaceutical composition is for inhibiting virus replication or transmission in a subject. In some embodiments, the virus is selected from HIV and simian immunodeficiency virus. In particular embodiments, the virus is Fuzeon-resistant.

According to another aspect, the present invention provides a method for inhibiting virus replication or transmission in a subject comprising administering to the subject in need of such treatment a therapeutically effective amount of the pharmaceutical composition comprising as an active ingredient a sphingolipid-peptide conjugate of the present invention, thereby inhibiting the virus replication or transmission.

In some embodiments, the subject is a human. In another embodiment, the virus is HIV. In yet another embodiment, the virus is Fuzeon-resistant HIV-1.

According to another aspect, the present invention provides a method for inhibiting infection of a cell by a virus comprising contacting the cell with an effective amount of a sphingolipid-peptide conjugate of the present invention, thereby inhibiting the infection of the cell.

According to another aspect, the present invention provides a method for inhibiting membrane protein assembly in a cell comprising contacting the cell with an effective amount of a sphingolipid-peptide conjugate of the present invention, thereby inhibiting the membrane protein assembly.

According to another aspect, the present invention provides use of the pharmaceutical compositions comprising as an active ingredient a sphingolipid-peptide conjugate disclosed herein, for the preparation of a medicament for inhibiting infection of a cell by a virus and/or for inhibiting virus replication or transmission in a subject. In some embodiments, the virus is selected from HIV and simian immunodeficiency virus. In particular embodiments, the virus is Fuzeon-resistant.

These and other embodiments of the present invention will be better understood in relation to the figures, description, examples, and claims that follow.

### BRIEF DESCRIPTION OF THE FIGURES

FIGS. 1A-C: Model for HIV membrane fusion and the structure of lipids investigated in this study. (A) HIV gp41 transmembrane protein has at least three major conformations during membrane fusion: the native non-fusogenic conformation, the pre-hairpin conformation, and the hairpin conformation. (B) A side view of the recent hairpin structure of gp41 (PDB ID: 2x7r). The trimeric inner coiled-coil of the N-helices is highlighted in red and the three packing C-helices are highlighted in blue. C, carboxyl terminus; N amino terminus. The investigated short domains within the central core of the protein are indicated: (i) the 17-mer conserved pocket sequence, termed N17 (SEQ ID NO:1), and (ii) the 19-mer N-helix binding sequence, termed DP19 (SEQ ID NO:3). (C) The chemical structure of the investigated hydrophobic conjugates: (i) the cellular moieties consisted of dihydrosphingosine (sphinganine) (compound 1), palmitic acid (compound 2), and cholesterol (compound 3) and (ii) the non-cellular moiety consisted of tocopherol (compound 4).

FIGS. 2A-D: Sphinganine exclusively endows antiviral activity to a short conserved gp41 N-peptide in different HIV fusion assays. Influence of sphinganine-N17 (▪), palmitic acid-N17 (Δ), cholesterol-N17 (▴), and tocopherol-N17 (□) on HIV fusion: (A) TZM-bl cells that were infected with the wild-type fully contagious HXB2 strain. (B) Jurkat T cells that were infected with pseudotyped LAI strain, and (C) TZM-bl target cells that were fused to HIV ENV-expressing effector cells. A representative fusion inhibitory curve is presented. (d) The IC₅₀ of sphinganine-N17 in different viral fusion systems is presented. Results represent the mean±standard deviation (s.d.), (n=3).

FIGS. 3A-F: The specificity of anti-HIV activity of sphingopeptides is determined by both the peptides and their lipid moiety. (A) Viruses were constructed to have the same HIV core with three different ENV: LAI (CXCR4 tropic), AD8 (CCR5 tropic) and vesicular stomatitis virus G (VSV-G). The three constructed viruses were allowed to infect TZM-bl cells in the presence of increasing concentrations of sphinganine-N17. Representative viral infection inhibitory curves are presented (n=2) for LAI (▴), AD8 () and VSV-G (▪). The corresponding IC₅₀ values of LAI and AD8 were 312 nM±83 nM and 403 nM±70 nM. (B) Sphingosine, a sphinganine analogue, was conjugated to the peptides (compound 5). (C) TZM-bl cells were infected with fully contagious HXB2 HIV-1 in the presence of increased concentrations of sphingosine-N17. A representative viral infection inhibitory curve is presented. The IC₅₀ was 208 nM±5 nM (n=2). (D) Helical wheel representation that demonstrates the interaction between the NHR (highlighted in gray) regions of HIV gp41 in the core structure of the hairpin conformation as observed in the crystal structures. The intermolecular association between the N-helices occur between positions a and d of the helical wheel. (E) Mutational analysis of N17 interactions. In the N17mut(a,d) mutant, the residues at positions a and d of N17 were replaced by residues at positions f and c, respectively, of the CHR. Residue numbers correspond to the HXB2 gp160 variant. In the scrambled N17 mutant, the residues of N17 were randomly reorganized. (f) Mutational analysis of N17 interactions abolished sphingopeptides antiviral activity. TZM-bl cells were infected with fully infectuous HXB2 HIV-1 in the presence of increasing concentrations of N17mut(a,d) (), sphinganine- N17mut(a,d) (◯), scrambled N17 (▪) and sphinganine-scrambled N17 (□).

FIG. 4A-E: Sphinganine potentiates the activity of N -and C-peptides in wild-type and Fuzeon-resistant viruses. (A) Designation and location of the peptides within the gp41 core. The complex between N36 from the N-helix and C34 from the C-helix resembles the central core and is highlighted. The short domains N17 and DP19 are located within the central core whereas the ShN17 extends from the core. Residue numbers correspond to the HXB2 gp160 variant. Panels (B)-(D) are viral infection inhibitory curves of the compounds as well as their corresponding IC₅₀ values. TZM-bl cells were infected with fully contagious HXB2 HIV in the presence of increased concentrations of the following compounds: (B) only sphinganine (▴), sphinganine-N17 (sphing-N17) (♦), and sphinganine-ShN17 (sphing-ShN17) (□). (C) N-terminal conjugated N17K (sphing-N17K) (▴) or C-terminal conjugated N17K (N17K-sphing) (Δ). (D) DP19K alone () and DP19K-sphinganine (DP19K-sphing) (◯). In panel e, the cells were infected with Fuzeon-resistant pseudotyped LAI strain with an escape mutation in the gp41 core (V38E). The fusion inhibitory curves of the drug Fuzeon () and sphing-N17 (◯) as well as their corresponding IC₅₀ values are presented. The IC₅₀ values are presented as mean±s.d., n≧3 (*p<0.05, **p<0.02).

FIG. 5A-B: Sphingopeptides strongly bind and accumulate in the cell membrane. (A) Sphinganine conjugated residual peptides within the cell sustain a potent inhibitory effect. C34, Fuzeon and sphing-N17 were pre-incubated with TZM-bl cells, followed by washing to remove unbound peptides and the addition of fully contagious viruses to start the infection. The IC50 of the peptide with or without washing the cells (IC50 1 or IC50 2, respectively) was calculated. Results are presented as the mean ratio of (IC50 1)/(IC50 2)±s.d. (n≧2), which reflects the fold increase in IC50 following cell washing. (B) The fusion inhibitory activity of sphing-N17 when it is pre-incubated with TZM-bl cells prior to the addition of fully contagious virus or when it is immediately added to a mixture of the virus with cells.

FIG. 6A-D: Chemical synthesis schemes of the hydrophobic moieties conjugated to the peptides. (A) Conjugation of sphinganine (compound 1). (B) Conjugation of palmitic acid (compound 2). (C) Conjugation of cholesterol (compound 3), and (D) Conjugation of tocopherol (compound 4). The synthesis procedure was performed on the resin (indicated with a gray circle) and is described for each compound in the section of Methods. N,N-Diisopropylethylamine (DIEA), Benzotriazol-1-yl-oxytripyrrolidinophosphonium hexafluorophosphate (PyBop), N-Methylmorpholine (NMM), Dimethylformamide (DMF) and Triethylamine (Et₃N).

### DETAILED DESCRIPTION OF THE INVENTION

The present invention provides lipophilic conjugates comprising an isolated peptide coupled to a sphingolipid moiety, the peptide corresponding to a fragment of the transmembrane protein HIV-1 gp41. The sphingolipid-peptide conjugates of the invention are capable of binding to the transmembrane protein thereby inhibiting the functional assembly of said transmembrane protein. Said conjugates of the present invention display anti-fusogenic and anti-viral activities and are thus useful for inhibiting various biological events associated with membrane protein assembly, especially HIV transmission to uninfected cells.

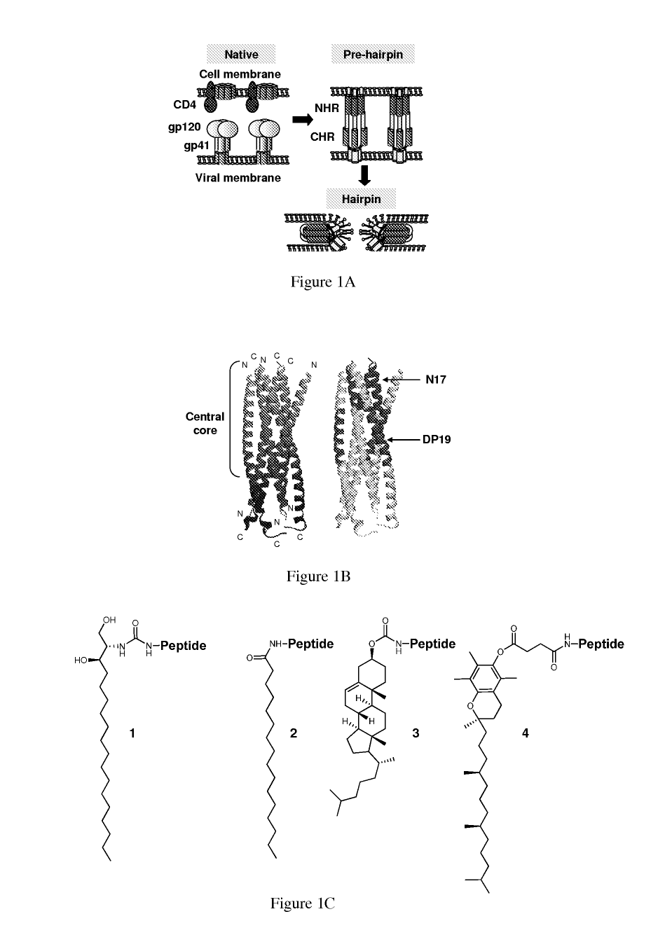

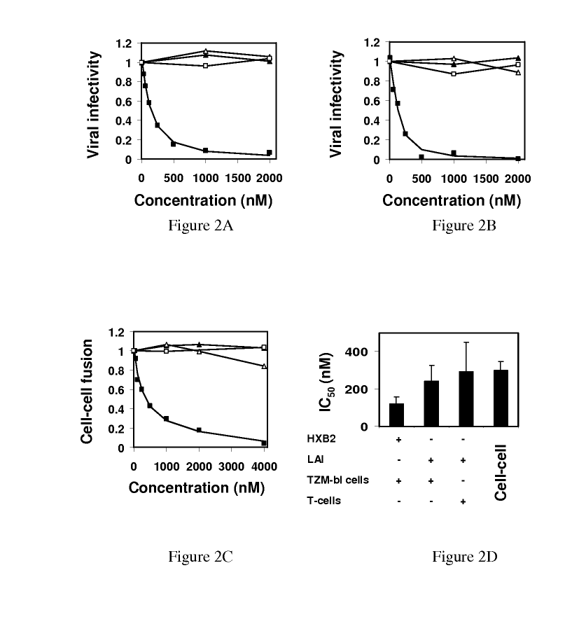

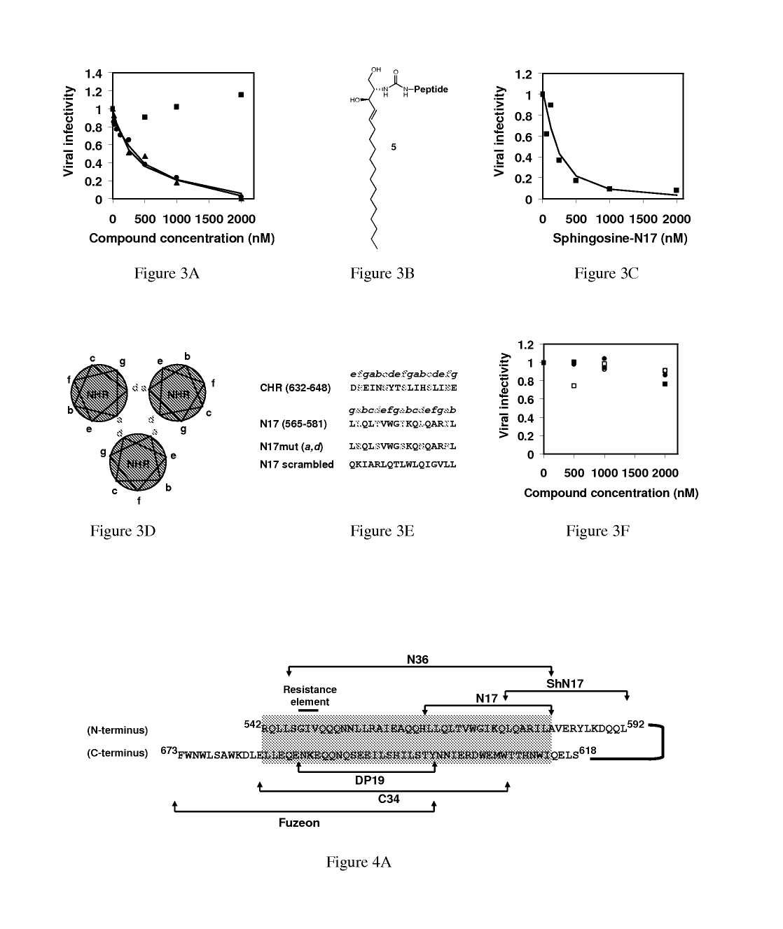

As demonstrated herein below, lipid moieties from different lipid classes were conjugated to short peptides from the HIV ENV core and their antiviral activities were investigated in several types of HIV infection assays (FIG. 1, FIG. 2). For the initial screening, the 17-mer pocket peptide from the gp41 core (termed N17; SEQ ID NO:1) was used, since it is highly conserved and is believed to facilitate some of the interactions within the core. Importantly, when the peptide was exogenously added, it was not active (Table 1), suggesting poor affinity to the viral fusion site. Unexpectedly, the only lipid moieties that potently amplified the antiviral activity of the peptide were sphinganine and its analogue sphingosine (Table 1, FIG. 2, FIG. 3C). No correlation was found between the antiviral activity and the hydrophobicity or the α-helical content of the peptide via the conjugation, as was previously reported for some peptide conjugates (Wexler-Cohen et al. Faseb J 24, 4196-4202, 2010; Eckert, D. M. & Kim, P. S. Proc Natl Acad Sci USA 98, 11187-11192, 2001).

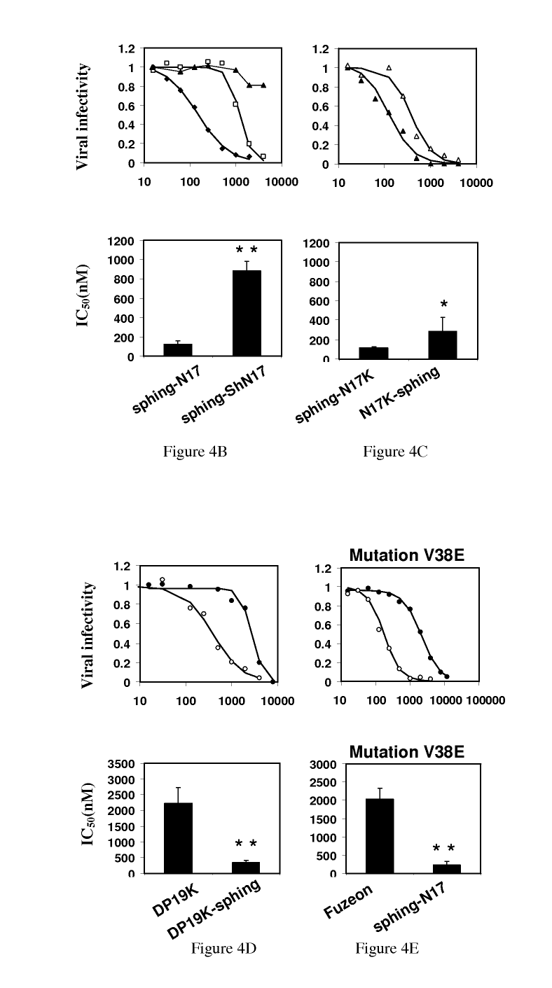

The analysis of the interplay between the sphingolipid backbone and its conjugated HIV peptide revealed that sphingopeptides have specific effect on HIV. This is based on the following reasons: first, sphingopeptides exhibited antiviral activity against HIV and not against vesicular stomatitis virus G (VSV-G) (FIG. 3A). Second, mutagenesis analysis of the peptides interactions by knocking out their ability to self-assemble or by reorganizing the sequence completely abrogated the antiviral activity of sphingopeptides (FIG. 3D-F). Third, sphinganine alone did not alter HIV infection at a concentration at which sphinganine-N17 completely blocked infection (FIG. 4B) and fourth, changing the sequence of the peptide, while keeping the same lipid backbone yielded different inhibitory levels (FIGS. 4B and 4D).

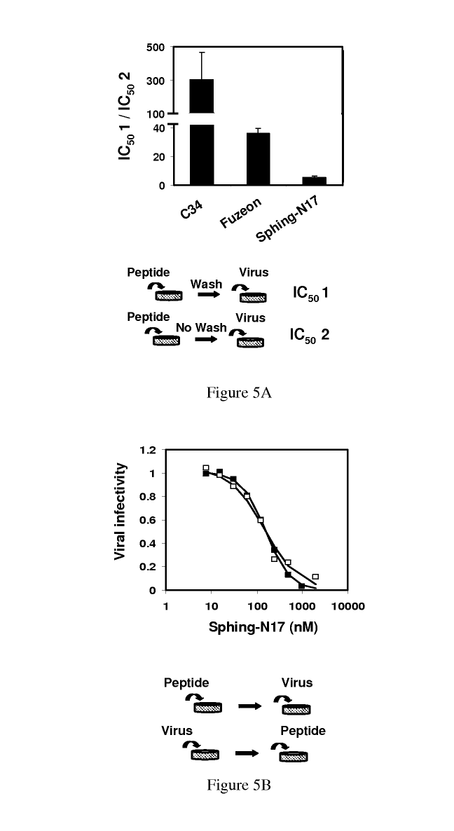

The viral membrane has a different lipid composition than does the cell membrane. This parameter may affect the types of lipids that are bound to the cell and the viral membranes during fusion. Without wishing to be bound by theory or mechanism of action, the sphingopeptides of the present invention are useful in inhibiting the cell membrane by (i) their fusion inhibitory activity on cell-cell fusion assays (FIG. 2C); (ii) the ability of sphinganine to preserve sufficient amounts of bound peptides within the target cells following washing (FIG. 5A) which prolonged their antiviral potencies; and (iii) the observation that no difference was found in the inhibitory curves of viral infectivity if the compound was added to target cells before adding the virus or immediately after adding it (FIG. 5B).

Studies have shown increased antiviral potency of N36 and C34 peptides derived from the ENV core when conjugated to fatty acid and cholesterol, respectively (Wexler-Cohen, Y. & Shai, Y. PLoS Pathog 5, e1000509, 2009; Ingallinella, P. et al. Proc Natl Acad Sci USA 106, 5801-5806, 2009). These studies used significantly long peptides (more than 30 residues) that by themselves are fusion inhibitors, suggesting that they already have affinity to the fusion site. Without wishing to be bound by theory or mechanism of action, the present invention showed for the first time that by minimizing the affinity of the peptides to the fusion site, while still keeping HIV specificity, sphingolipids are much stronger site-specific lipids.

As exemplified herein below, sphinganine and sphingosine exclusively endow strong antiviral activity to otherwise poorly or non-active short C- and N-peptides from the ENV core (Tables 2 and 3) in a broad spectrum of infection assays including Fuzeon-resistant viruses. The data support the recruitment of sphingopeptides to the HIV membrane fusion site with enhanced membrane-binding affinity that prolonged their inhibitory potencies. The sphingopeptides conjugates may also extend the half-life time of fusion inhibitors in-vivo and as topical blockers of viral transmission during sexual intercourse. To the best of our knowledge, sphingopeptides are the shortest lipid-based HIV peptides that potently inhibit viral fusion. This is important since the cost of manufacturing peptides for therapeutic applications dramatically rises with their increasing length. Furthermore, sphingolipids are involved in many cellular processes such as signaling, viral infection and membrane trafficking, and therefore the present invention suggest a general concept in developing inhibitors to sphingolipid-mediated biological systems.

According to some embodiments, the present invention provides sphingolipid-peptide conjugates (also termed herein “sphingopeptides”) comprising an isolated peptide of no more than 22 amino acid residues derived from HIV gp41 core structure, coupled to a sphingolipid moiety. In a particular embodiment, the peptide comprises no more than 22 amino acid residues corresponding to a fragment of amino acids 542 to 592 of HIV-1_{HXB2} gp160 (SEQ ID NO: 13) or an analog, a derivative or fragment thereof. In another particular embodiment, the peptide comprises no more than 22 amino acid residues corresponding to a fragment of amino acids 618 to 673 of HIV-1_{HXB2} gp160 (SEQ ID NO: 14), or an analog, a derivative or fragment thereof.

The terms “sphingolipid-peptide conjugates” “lipophilic conjugate” and “lipopeptide conjugate” used interchangeably throughout the specification designate a conjugate comprising a peptide covalently coupled to a sphingolipid.

According to another embodiment, the present invention provided sphingolipid-peptide conjugate having the formula (I):

X₁-X₂-L-Q-L-T-X₃-W-G-I-K-Q-L-X₄-A-R-I-L-X₅-X₆ (I); wherein X₁ or X₆ is a sphingolipid, X₂ is L or E, X₃ is V or Q, X₄ is Q or L; and X₅ is K or absent. In some embodiments, the peptide is an analog, a derivative or fragment of the amino acid sequence provided in formula (I).

According to yet additional embodiments, the sphingolipid-peptide conjugate is selected from the group consisting of:

Sphinganine-LLQLTVWGIKQLQARIL;

Sphingosine-LLQLTVWGIKQLQARIL;

Sphinganine-LLQLTVWGIKQLQARILK;

Sphingosine-LLQLTVWGIKQLQARILLK;

LLQLTVWGIKQLQARILK-Sphinganine;

LLQLTVWGIKQLQARILLK-Sphingosine;

Sphinganine-ELQLTQWKIKQLLARIL;

Sphingosine-ELQLTQWKIKQLLARIL;

Sphinganine-ELQLTQWKIKQLLARILK;

Sphingosine-ELQLTQWKIKQLLARILK;

ELQLTQWKIKQLLARILK-Sphinganine;

ELQLTQWKIKQLLARILK-Sphingosine;

Sphinganine-LLQLTVWGIKQLQA;

Sphingosine-LLQLTVWGIKQLQA;

Sphinganine-YTSLIHSLIEESQNQQEKN

Sphingosine-YTSLIHSLIEESQNQQEKN

YTSLIHSLIEESQNQQEKNK-Sphinganine;

YTSLIHSLIEESQNQQEKN-Sphingosine;

Sphinganine-TTWMEWDREINNYT;

Sphingosine-TTWMEWDREINNYT;

Sphinganine-TTWMEWDREINNYTK;

Sphingosine-TTWMEWDREINNYTK;

TTWMEWDREINNYTK-Sphinganine;

TTWMEWDREINNYTK-Sphingosine;

TWMEWDREIK-Sphinganine;

IwMewDREIK-Sphinganine (“w” denotes D-Trp and “e” denotes D-Glu);

Sphinganine-LQARILAVERYLKDQQL;

Sphingosine-LQARILAVERYLKDQQL;

Each possibility is a separate embodiment of the present invention.

According to particular embodiments the peptide is selected from the group consisting of:

LLQLTVWGIKQLQARIL (SEQ ID NO:1);

LLQLTVWGIKQLQARILK (SEQ ID NO:5);

ELQLTQWKIKQLLARIL (SEQ ID NO:6);

ELQLTQWKIKQLLARILK (SEQ ID NO:7); and

LLQLTVWGIKQLQA (SEQ ID NO:8);

According to another particular embodiment, the peptide is selected from the group consisting of:

YTSLIHSLIEESQNQQEKN (SEQ ID NO:3); and

YTSLIHSLIEESQNQQEKNK (SEQ ID NO:9);

According to some embodiments, the peptide comprises the amino acid sequence as set forth in TWMEWDREI (SEQ ID NO:15). According to yet another particular embodiment, the peptide is selected from the group consisting of:

TTWMEWDREINNYT (SEQ ID NO: 4);

TTWMEWDREINNYTK (SEQ ID NO:10);

TWMEWDREI (SEQ ID NO:15);

TWMEWDREIK (SEQ ID NO:11); and

IwMewDREIK (SEQ ID NO:12) wherein “w” denotes D-Trp and “e” denotes D-Glu).

According to an exemplary embodiment, the peptide is LQARILAVERYLKDQQL (SEQ ID NO:2).

According to another embodiment, the present invention provides an isolated peptide selected from the group consisting of SEQ ID NO: 2, 5, 6, 7, 8, 9, 10, 11, 12 and 15. It should be understood that the peptides according to the principles of the present invention do not include any known peptides.

According to the principles of the present invention, said sphingolipid-peptide conjugate is capable of inhibiting protein-induced membrane fusion.

The term “membrane binding” of the conjugate of the invention refers to a peptide capable of interacting or binding to membranal lipids.

The terms “membrane protein assembly” or “functional assembly” of a transmembrane protein is used herein to refer to complex formation or non-covalent interaction between transmembrane proteins, which lead to membrane fusion events and/or to intracellular processes initiated by the membrane protein complex formation or membrane protein interactions. The term “membrane protein” is used herein to refer to cellular membrane proteins of human or non-human cells as well as to viral envelope proteins. It should be understood that functional assembly of a protein includes homodimerization and heterodimerization, i.e., the protein may interact with an identical protein or it may interact with a different protein. Thus, functional assembly includes, but is not limited to, an interaction between two proteins adjacent to each other to form a non-covalent complex within the same cellular membrane and an interaction between different membrane proteins present in different cells. The terms “functional assembly of a membrane protein” and “membrane protein assembly” are used interchangeably. The term “transmembrane protein” refers to a membrane protein that spans the lipid bilayer of the membrane.

According to the principles of the present invention, the isolated peptide prior to conjugation of the sphingolipid moiety is either inactive or with lesser antiviral activity. Conjugation of the sphingolipid moiety endows the peptide with an antiviral activity so that the activity is significantly higher after conjugation than prior to conjugation. According to some embodiments, conjugation of a sphingolipid moiety to a peptide of the invention enhances the antiviral activity by at least 2 fold. According to some other embodiments, conjugation of a sphingolipid moiety to a peptide of the invention enhances the antiviral activity by at least 10 fold. According to some other embodiments, conjugation of a sphingolipid moiety to a peptide of the invention enhances the antiviral activity by at least 20 fold.

As demonstrated herein, the sphingolipids exclusively endowed nanomolar antiviral activity to specific peptides derived from HIV-1 gp41 NHR or CHR regions. As used herein, the term “nanomolar antiviral activity” relates to the having an IC₅₀ of about 50-500 nM, particularly 100-400 nM. In exemplary embodiments, the sphingolipids exhibit antiviral activity having an IC₅₀ as low as about 100-250 nM.

According to some embodiments, the hydrophobic moiety may be coupled to the peptide through any other free functional group along the peptide chain, for example, to the ε-amino group of lysine. According to some embodiments, the hydrophobic moiety may be coupled to the peptide through chemical spacer or linker. According to further embodiments, more than one hydrophobic moiety may be coupled to the peptide, through the N-terminus, C-terminus or through any other functional group along the peptide chain. Each possibility represents a separate embodiment of the present invention.

According to certain embodiments of the invention, the isolated peptide comprises up to 22 amino acid residues, up to 21 amino acid residues, up to 20 amino acid residues, up to 19 amino acid residues, up to 18 amino acid residues, up to 17 amino acid residues, up to 16 amino acid residues, up to 15 amino acid residues, up to 14 amino acid residues, up to 13 amino acid residues, up to 12 amino acid residues, up to 11 amino acid residues or up to 11 amino acid residues, wherein each possibility is a separate embodiment of the present invention.

According to other embodiments of the invention, the isolated peptide comprises at least 7 amino acid residues, at least 8 amino acid residues, at least 9 amino acid residues or at least 10 amino acid residues, wherein each possibility is a separate embodiment of the present invention.

According to other embodiments of the invention, the isolated peptide comprises 10-22 amino acid residues, 10-21 amino acid residues, 10-20 amino acid residues, 10-19 amino acid residues, 10-18 amino acid residues or 10-17 amino acid residues, wherein each possibility is a separate embodiment of the present invention.

It is to be understood that within the scope of the present invention are peptide derivatives, analogs or salts thereof conjugated to a sphingolipid moiety according to embodiments of the present invention, wherein the derivative, analog or salt thereof displays anti-fusogenic activity when conjugated to the sphingolipid moiety.

The present invention encompasses sphingopeptide derivatives and analogs having amino acid substitutions, and/or extensions.

The term “analog” as used herein refers to peptides according to embodiments of the invention comprising altered sequences by amino acid substitutions or chemical modifications. The amino acid substitutions may be of conserved or non-conserved nature. Conserved amino acid substitutions consist of replacing one or more amino acids of an all L-amino acid or diastereomeric peptide of the invention with amino acids of similar charge, size, and/or hydrophobicity characteristics. For example, one or more amino acid residues within the sequence can be substituted by another amino acid of a similar polarity, which acts as a functional equivalent, resulting in a silent alteration. Substitutes for an amino acid within the sequence may be selected from other members of the class to which the amino acid belongs. For example, the non-polar (hydrophobic) amino acids include alanine, leucine, isoleucine, valine, proline, phenylalanine, tryptophan and methionine. The polar neutral amino acids include glycine, serine, threonine, cysteine, tyrosine, asparagine, and glutamine. The positively charged (basic) amino acids include arginine, lysine and histidine. The negatively charged (acidic) amino acids include aspartic acid and glutamic acid. Such substitutions are known as conservative substitutions. Non-conserved substitutions consist of replacing one or more amino acids of an all L-amino acid or a diastereomeric peptide with amino acids possessing dissimilar charge, size, and/or hydrophobicity characteristics, such as, for example, substitution of a glutamic acid (E) to valine (V). The amino acid substitutions may also include non-natural amino acids.

In some embodiments, said analogs of the invention have at least 70%, at least 75% or at least 80% identity to the isolated peptides disclosed herein. It will be appreciated by a skilled person in the art that SEQ ID NO:6 is an analog of SEQ ID NO:1 and SEQ ID NO:7 is an analog of SEQ ID NO:5.

Amino acid extensions may consist of a single amino acid residue or stretches of residues. The extensions may be made at the carboxy or amino terminal end of peptides of the invention. Such extensions will generally range from 2 to 17 amino acids in length. Preferably, the peptide comprises not more than 22 amino acid residues in total. One or more such extensions may be introduced into a peptide so long as such extensions result in a peptide, which still exhibits anti-fusogenic activity by itself or when conjugated to a hydrophobic moiety. According to some preferred embodiments, the extensions of the peptides of the invention comprise at least one positively charged amino acid at the amino terminus, at the carboxy terminus, or at both termini of the peptide. Positively charged amino acids that may be added to the peptides of the invention include, but are not limited to, lysine, arginine, histidine, or any other non-charged amino acid derivatized to yield a positively charged amino acid.

Typically, the present invention encompasses derivatives of the sphingopeptides of the invention. The term “derivative” includes any chemical derivative of the peptide having one or more residues chemically derivatized by reaction of side chains or functional groups. Such derivatized molecules include, for example, those molecules in which free amino groups have been derivatized to form amine hydrochlorides, p-toluene sulfonyl groups, carbobenzoxy groups, t-butyloxycarbonyl groups, chloroacetyl groups or formyl groups. Free carboxyl groups may be derivatized to form salts, methyl and ethyl esters or other types of esters or hydrazides. Free hydroxyl groups may be derivatized to form O-acyl or O-alkyl derivatives. The imidazole nitrogen of histidine may be derivatized to form N-im-benzylhistidine. Also included as chemical derivatives are those peptides, which contain one or more naturally occurring amino acid derivatives of the twenty standard amino acid residues. For example: 5-hydroxylysine may be substituted for lysine; 3-methylhistidine may be substituted for histidine; homoserine may be substituted or serine; and ornithine may be substituted for lysine. The term “derivative” may further include chemical derivatives of the fatty acid moieties.

The present invention provides sphingopeptides comprising a peptide which comprises from about 7 to 22 amino acid residues corresponding to a fragment of a transmembrane protein, particularly of HIV gp41 protein. According to some embodiments, the peptides comprise the amino acid sequence of a transmembrane domain of a membrane protein.

According to the invention, the sphingopeptides exhibit inhibitory activity of functional assembly of a membrane protein. The inhibitory activity of functional assembly of a membrane protein includes, but is not limited to, anti-fusogenic activity and anti-viral activity.

The terms “anti-fusogenic” and “anti-membrane fusion” and “cell fusion inhibitor”, as used herein, refer to an agent's ability to inhibit or reduce the level of membrane fusion events between two or more moieties relative to the level of membrane fusion which occurs between these moieties in the absence of the sphingopeptide of the invention. The moieties may be, for example, cell membranes or viral structures, such as viral envelopes or pili. The term “anti-viral”, as used herein, refers to the compound's ability to inhibit viral infection of cells, via, for example, cell-cell fusion or free virus infection. Such infection may involve membrane fusion, as occurs in the case of enveloped viruses, or some other fusion event involving a viral structure and a cellular structure (e.g., such as the fusion of a viral pilus and bacterial membrane during bacterial conjugation). A sphingopeptide of the invention exhibits an anti-fusogenic and/or anti-viral activities if the level of membrane fusion events is lower in the presence of the sphingopeptide than in its absence.

Assays for cell fusion events are well known to those of skill in the art. Cell fusion assays are generally performed in vitro. Such an assay includes culturing cells, which, in the absence of any treatment, would undergo an observable level of syncytial formation. For example, uninfected cells may be incubated in the presence of cells chronically infected with a virus that induces cell fusion. Viruses that induce cell fusion include, but are not limited to, HIV, SIV, or respiratory syncytial virus.

For the cell fusion assay, cells are incubated in the presence of a sphingopeptide to be assayed. For each sphingopeptide, a range of sphingopeptide concentrations may be tested. This range should include a control culture wherein no sphingopeptide has been added.

Standard conditions for culturing cells, well known to those of ordinary skill in the art, are used. After incubation for an appropriate period, the culture is examined microscopically for the presence of multinucleated giant cells, which are indicative of cell fusion and syncytial formation. Well-known stains, such as crystal violet stain, may be used to facilitate the visualization of syncytial formation. Alternatively or additionally, cell fusion may be detected by fluorescent dye transfer between labeled donor cells such as, for example, cells expressing HIV-1 gp120-41 and acceptor cells such as, for example, mouse fibroblasts, labeled with a different fluorescent dye. Addition of a sphingopeptide of the present invention inhibits dye transfer, which is indicative of inhibition of cell fusion. Another example comprised cell-lines of human T-cells, such as Jurkat E6-1 and Jurkat HXBc2 cells. Jurkat HXBc2 cells express HIV-1 HXBc2 Rev and ENV proteins, whereas Jurkat E6-1 are normal T-cells. Each cell type is labeled with either DiI or DiD lipophilic fluorescent probes, respectively. The two cell populations are co-incubated in the presence of different concentrations of the inhibitory sphingopeptide. The percentage of fused cells, with or without the peptides, is collected using flow cytometry and upgraded to a FACSCalibur cell analyzer.

Other assay to evaluate the inhibitory activity of a sphingopeptide in membrane protein assembly may use the ToxR system, which is a robust method for detecting homodimerization of transmembrane domains in vivo.

Assays to test anti-viral activities of a sphingopeptide may be based upon measuring an enzymatic activity of a virus as a function of viral infection. If taking HIV as an example, a reverse transcriptase (RT) assay may be utilized to test a lipopeptide ability to inhibit infection of CD4+ cells by cell-free HIV. Such an assay may comprise culturing an appropriate concentration (i.e., TCID50) of virus and CD4+ cells in the presence of the lipopeptide to be tested. Culture conditions well known to those in the art are used. A range of sphingopeptide concentrations may be used, in addition to a control culture wherein no sphingopeptide has been added. After incubation for an appropriate period of culturing, a cell-free supernatant is prepared, using standard procedures, and tested for the presence of RT activity as a measure of successful infection. The RT activity may be tested using standard techniques (see Goff, S. et al., 1981, J. Virol. 38:239-248; Willey, R. et al., 1988, J. Virol. 62:139-147). Another assay to test anti-viral activities of a lipopeptide may be based upon measuring luciferase activity in cells infected with the viruses in the presence of the sphingopeptide. These assays normally comprised CD4+ and co-receptor expressing cells, such as TZM-bl Hela cells. In addition, these cells contain a reporter luciferase gene which is expressed upon induction by viral proteins in infected cells. The luminescence signal in cells is decreased when incubating the inhibitory sphingopeptide with the virus-cell mixture.

Standard methods, which are well known to those of skill in the art, may be utilized for assaying non-retroviral activity. See, for example, Pringle et al. (Pringle, C. R. et al., 1985, J. Medical Virology 17:377-386) for a discussion of respiratory syncytial virus and parainfluenza virus activity assay techniques.

In vivo assays may also be utilized to test, for example, the antiviral activity of the sphingopeptide of the invention. To test for anti-HIV activity, for example, the in vivo model described in Barnett et al. may be used (Barnett, S. W. et al., 1994, Science 266:642-646, the content of which is incorporated by reference as if fully set forth herein).

The anti-fusogenic capability of the sphingopeptides of the invention may additionally be utilized to inhibit or treat/ameliorate symptoms caused by processes involving membrane fusion events. Such events may include, for example, virus transmission via cell-cell fusion, and sperm-egg fusion. Further, the sphingopeptides of the invention may be used to inhibit free viral infection or transmission of uninfected cells wherein such viral infection involves cell-cell fusion events or involves fusion of a viral structure with a host cell membrane.

Retroviral viruses whose transmission may be inhibited by the sphingopeptides of the invention include, for example, human retroviruses, particularly HIV-1 and HIV-2.

The anti-viral activity of the sphingopeptides of the invention may show a pronounced type and subtype specificity, i.e., specific sphingopeptides may be effective in inhibiting the activity of only specific viruses. This feature of the invention presents many advantages. One such advantage, for example, lies in the field of diagnostics, wherein one can use the antiviral specificity of the lipopeptide of the invention to ascertain the identity of a viral isolate.

The peptides of the present invention can be synthesized using methods well known in the art including chemical synthesis and recombinant DNA technology. Synthesis may be performed by solid phase peptide synthesis described by Merrifield (see J. Am. Chem. Soc., 85:2149, 1964). Alternatively, the peptides of the present invention can be synthesized using standard solution methods (see, for example, Bodanszky, M., Principles of Peptide Synthesis, Springer-Verlag, 1984). Preferably, the peptides of the invention are synthesized by solid phase peptide synthesis.

The invention further contemplates sphingolipid conjugates comprising peptides composed of all L-amino acids or diasteriomeric peptides. The term “diastereomeric peptide” as used herein refers to a peptide comprising both L-amino acid residues and D-amino acid residues. When there is no indication, the amino acid residue occurs in L isomer configuration. In an exemplary embodiment, the diastereomeric peptide is IwMewDREIK (SEQ ID NO: 12), wherein “w” denotes D-Trp and “e” denotes D-Glu.

Positively charged amino acids as used herein are selected from positively charged amino acids known in the art. Examples of positively charged amino acids are lysine, arginine, and histidine. Hydrophobic amino acids as used herein are selected from hydrophobic amino acids known in the art. Examples of hydrophobic amino acids are leucine, isoleucine, glycine, alanine, and valine. Negatively charged amino acids are selected from negatively charged amino acids known in the art including, but not limited to, glutamic acid and aspartic acid.

### Sphingolipids

The term “sphingolipid” as used herein means a natural and synthetic substance comprising a long-chain base (LCB) (i.e. sphingoid base, a long-chain hydrocarbon material derived from d-erythro-2-amino-1,3-diol), generally comprising a polar head group. In one embodiment, said sphingolipid is a sphingoid base. In another embodiment, said sphingolipid is a sphingoid base selected from the group of sphinganines, sphingosines or phytosphingosines. The term “sphingoid base” as used herein refers to long chain amino alcohols that may differ in length of the alkyl chain lengths and extend of branching. The most common long-chain bases in mammals are sphingosine, sphinganine and phytosphingosine.

Non-limiting examples of the sphingoids or sphingoid bases which may be used in the contents of the present invention include sphingosine, dihydrosphingosine, phytosphingosine, dehydrophytosphinosine and derivatives thereof. Non-limiting examples of such derivatives include acyl derivatives, such as ceramide (N-acylsphingosine), dihydroceramides, phytoceramides and dihydrophytoceramides as well as ceramines (N-alkylsphinogsines) and the corresponding derivatives (e.g. dihydroceramine, phytoceramine, dihydrophytoceramines etc.). According to additional embodiments, said sphingolipid is selected from sphingomyelin, ceramide and cerebroside.

The sphingolipid may also be provided as a mixture of different sphingolipids. In another embodiment, said sphingolipid may further comprise an amide-linked fatty acid, such as compounds generally referred to as “lysosphingolipids”.

Sphinganine and sphingosine are simple sphingolipids that serve as building blocks of more complex sphingolipids (Futerman, A. H. & Riezman, H. Trends Cell Biol 15, 312-318, 2005; Goni, F. M. & Alonso, A. Biochim Biophys Acta 1758, 1902-1921, 2006). Sphinganine is a polar lipid comprising hydroxyl groups (FIG. 1C) and could favor its interaction with the sphingolipid backbone chains through hydrogen bonds.

According to some embodiments of the present invention, the sphingolipid may be coupled to the N-terminal, to the C-terminal, or to any other free functional group along the peptide chain, for example, to the ε-amino group of lysine. It should be understood that the sphingolipid is covalently coupled to the peptide. The terms “coupling” and “conjugation” are used herein interchangeably and refer to the chemical reaction, which results in covalent attachment of a sphingolipid to a peptide to yield a lipophilic conjugate. Coupling of a sphingolipid to a peptide is performed similarly to the coupling of an amino acid to a peptide during peptide synthesis. Alternatively, the coupling of a sphingolipid to a peptide may be performed by any coupling method known in the art.

### Pharmaceutical Composition

The present invention provides pharmaceutical compositions comprising the sphingopeptide of the invention and a cosmetically and/or pharmaceutically acceptable carrier. The term “pharmaceutically acceptable carrier” refers to a vehicle which delivers the active components to the intended target and which does not cause harm to humans or other recipient organisms. As used herein, “pharmaceutical” will be understood to encompass both human and animal pharmaceuticals. Useful carriers include, for example, water, acetone, ethanol, ethylene glycol, propylene glycol, butane-1,3-diol, isopropyl myristate, isopropyl palmitate, or mineral oil. Methodology and components for formulation of pharmaceutical compositions are well known, and can be found, for example, in Remington's Pharmaceutical Sciences, Eighteenth Edition, A. R. Gennaro, Ed., Mack Publishing Co. Easton Pa., 1990. The pharmaceutical composition may be formulated in any form appropriate to the mode of administration, for example, solutions, colloidal dispersions, emulsions (oil-in-water or water-in-oil), suspensions, creams, lotions, gels, foams, sprays, aerosol, ointment, tablets, suppositories, and the like.

The pharmaceutical compositions can also comprise other optional materials, which may be chosen depending on the carrier and/or the intended use of the composition. Additional components include, but are not limited to, antioxidants, chelating agents, emulsion stabilizers, e.g., carbomer, preservatives, e.g., methyl paraben, fragrances, humectants, e.g., glycerin, waterproofing agents, e.g., PVP/Eicosene Copolymer, water soluble film-formers, e.g., hydroxypropyl methylcellulose, oil-soluble film formers, cationic or anionic polymers, and the like.

The pharmaceutical compositions useful in the practice of the present invention comprise a sphingopeptide of the invention optionally formulated into the pharmaceutical composition as a pharmaceutically acceptable salt form. Pharmaceutically acceptable salts include the acid addition salts (formed with the free amino groups of the polypeptide), which are formed with inorganic acids, such as for example, hydrochloric or phosphoric acid, or with organic acids such as acetic, oxalic, tartaric, and the like. Suitable bases capable of forming salts with the lipopeptides of the present invention include, but are not limited to, inorganic bases such as sodium hydroxide, ammonium hydroxide, potassium hydroxide and the like; and organic bases such as mono-, di- and tri-alkyl and aryl amines (e.g. triethylamine, diisopropyl amine, methyl amine, dimethyl amine and the like) and optionally substituted ethanolamines (e.g. ethanolamine, diethanolamine and the like).

The anti-fusogenic capability of the short sphingopeptide of the invention may additionally be utilized to inhibit or treat/ameliorate symptoms caused by processes involving membrane fusion events. Such events may include, for example, virus transmission via cell-cell fusion, and sperm-egg fusion. Further, the short lipopeptides of the invention may be used to inhibit free viral infection or transmission of uninfected cells wherein such viral infection involves cell-cell fusion events or involves fusion of a viral structure with a host cell membrane.

Retroviral viruses whose transmission may be inhibited by the short lipopeptides of the invention include, for example, human retroviruses, particularly HIV, even more particularly HIV-1.

One such advantage, for example, lies in the field of diagnostics, wherein one can use the anti-fusogenic specificity of the lipopeptide of the invention to ascertain the identity of a viral isolate.

According to another aspect, the present invention provides a pharmaceutical composition comprising a therapeutically effective amount of a sphingopeptide according to the principles of the present invention and a pharmaceutically acceptable carrier, the lipophilic conjugate capable of inhibiting fusion of a transmembrane protein, without wishing to be bound by theory or mechanism of action, the anti-fusogenic activity of the lipopeptides of the invention originated from their ability to interfere with the functional assembly of a viral transmembrane protein.

A pharmaceutical composition useful in the practice of the present invention typically contains a sphingopeptide of the invention formulated into the pharmaceutical composition as a pharmaceutically acceptable salt form. Pharmaceutically acceptable salts may be prepared from pharmaceutically acceptable non-toxic acids, including inorganic and organic acids. Such acids include acetic, benzenesulfonic, benzoic, camphorsulfonic, citric, ethanesulfonic, fumaric, gluconic, glutamic, hydrobromic, hydrochloric, isethionic, lactic, maleic, malic, mandelic, methanesulfonic, mucic, nitric, pamoic, pantothenic, phosphoric, succinic, sulfuric, tartaric, p-toluenesulfonic acid, and the like.

Pharmaceutically acceptable salts may be prepared from pharmaceutically acceptable non-toxic bases including inorganic or organic bases. Salts derived from inorganic bases include aluminum, ammonium, calcium, copper, ferric, ferrous, lithium, magnesium, manganic, manganous, potassium, sodium, zinc, and the like. Salts derived from pharmaceutically acceptable organic non-toxic bases include salts of primary, secondary, and tertiary amines, substituted amines including naturally occurring substituted amines, cyclic amines, and basic ion exchange resins, such as arginine, betaine, caffeine, choline, N,N′-dibenzylethylenediamine, diethylamine, 2-diethylaminoethanol, 2-dimethylaminoethanol, ethanolamine, ethylenediamine, N-ethyl-morpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine, and the like.

A therapeutically effective amount of a lipophilic conjugate of the invention is an amount that when administered to a patient is capable of exerting an inhibitory activity of functional assembly of a membrane protein and hence of membrane fusion events such as, for example, viral infection, bacterial infection, and intracellular processes involving protein membrane assembly. According to some embodiments, a pharmaceutical composition of the present invention is useful for inhibiting a viral disease in a patient as described further herein. According to such embodiments, a therapeutically effective amount is an amount that when administered to a patient is sufficient to inhibit, preferably to eradicate, a viral disease.

The pharmaceutical compositions of the present invention comprise at least one sphingopeptide conjugate according to the present invention, and methods of the present invention involve the administration of at least one lipophilic conjugate according to the present invention.

It is to be further understood that the sphingopeptide of the invention may be therapeutically used in combination with additional peptides and lipopeptides that target different sequences along the transmembrane protein. For example, a short sphingopeptide of the invention derived from the N-terminus of HIV-1 gp41 NHR, can work together with a peptide derived from the HIV-1 gp41 CHR sequence targeting the pocket region. Peptides and lipopeptides comprising sequences that cannot bind each other can potentially be combined. Such sequences would not neutralize each other's effect but rather enhance it.

Since most research so far has been concentrated on targeting peptides to interfere with the formation of known pocket regions (e.g. DP178, DP107 and even N36), having short-lipopeptides of less than 23 amino acid residues that target different sequences along the HIV-1 gp41 would be advantageous.

The preparation of pharmaceutical compositions, which contain peptides as active ingredients, is well known in the art. Typically, such compositions are prepared as injectable, either as liquid solutions or suspensions. However, solid forms, which can be suspended or solubilized prior to injection, can also be prepared. The preparation can also be emulsified. The active therapeutic ingredient is mixed with inorganic and/or organic carriers, which are pharmaceutically acceptable and compatible with the active ingredient. Carriers are pharmaceutically acceptable excipients (vehicles) comprising more or less inert substances that are added to a pharmaceutical composition to confer suitable consistency or form to the composition. Suitable carriers are, for example, water, saline, dextrose, glycerol, ethanol, or the like and combinations thereof. In addition, if desired, the composition can contain minor amounts of auxiliary substances such as wetting or emulsifying agents, pH buffering agents, stabilizers, and anti-oxidants, which enhance the effectiveness of the active ingredient.

The pharmaceutical composition can be delivered by a variety of means including intravenous, intramuscularly, infusion, intranasal, intraperitoneal, subcutaneous, rectal, topical, or into other regions, such as into synovial fluids. However delivery of the composition transdermally is also contemplated, such by diffusion via a transdermal patch.

According to another aspect the present invention provides a method for inhibiting membrane protein assembly in a cell comprising contacting the cell with an effective amount of a membrane binding lipophilic conjugate according to the principles of the present invention, thereby inhibiting membrane protein assembly.

According to a further aspect, the present invention provides a method for inhibiting infection of a cell by a virus comprising contacting the cell with an effective amount of a membrane binding lipophilic conjugates according to the principles of the present invention, thereby inhibiting the infection of the cell.

According to still a further aspect, the present invention provides a method for inhibiting virus replication and transmission in a subject comprising administering to the subject a therapeutically effective amount of a pharmaceutical composition comprising a lipophilic conjugate according to the principles of the present invention dispersed in a pharmaceutically acceptable carrier or diluent.

Patients in which the inhibition of viral replication would be clinically useful include patients suffering from diseases transmitted by various viruses including, for example, human retroviruses, particularly HIV-1 and HIV-2.

The pharmaceutical composition is administered in a manner compatible with the dosage formulation, and in a therapeutically effective amount. The quantity to be administered depends on the subject to be treated, and the capacity of the subject's blood hemostatic system to utilize the active ingredient. Precise amounts of active ingredient required to be administered depend on the judgment of the practitioner and are peculiar to each individual.

Methods of treating a disease according to the invention may include administration of the pharmaceutical compositions of the present invention as a single active agent, or in combination with additional methods of treatment. The methods of treatment of the invention may be in parallel to, prior to, or following additional methods of treatment. Methods of treating a disease according to the invention may include administration of the pharmaceutical compositions of the present invention as a single active agent, or in combination with additional methods of treatment. The methods of treatment of the invention may be in parallel to, prior to, or following additional methods of treatment.

The following examples are presented in order to more fully illustrate some embodiments of the invention. They should, in no way be construed, however, as limiting the broad scope of the invention.

### EXAMPLES

Materials and Methods.

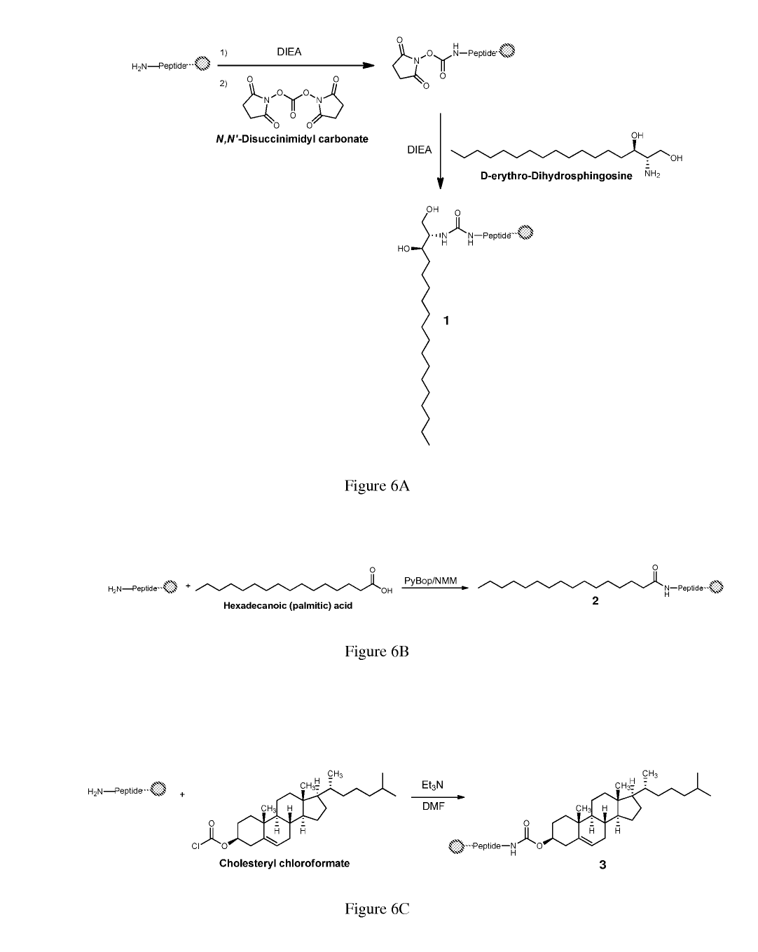

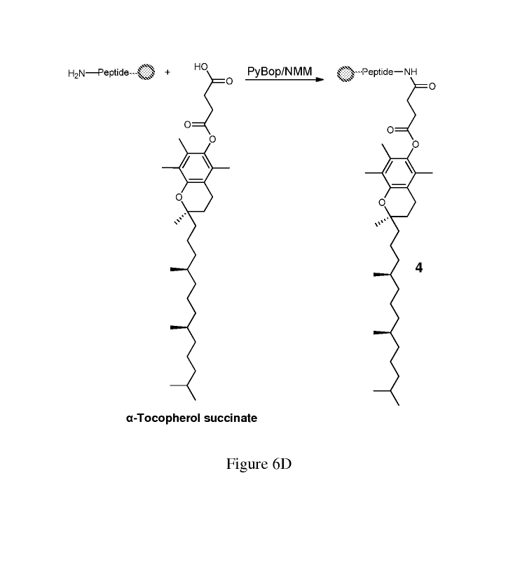

Peptide synthesis, lipid moiety conjugation, and fluorescent labeling. Peptides were synthesized on Rink Amide MBHA resin by using the F-moc strategy. Several peptides contain a lysine residue at their C-terminus with an MTT side-chain protecting group (Nova-biochem AG, Switzerland) that requires a special deprotection step under mild acidic conditions (2×1 min of 5% trifluoroacetic acid (TFA) in dichloromethane (DCM) and 30 min of 1% TFA in DCM). This enables the conjugation of a lipid moiety or a fluorescent probe to the C-terminus. The chemical synthesis schemes of the following compounds 1-5 are described in FIG. 6. Conjugation of hexadecanoic (palmitic) acid (C16) or α-Tocopherol succinate (Sigma Chemical Co., Israel) to the N-terminus of selected peptides (compound 2 and compound 4, respectively) was performed using standard F-moc chemistry. Conjugation of cholesterol to the N-terminus of a peptide (compound 3) was performed by adding 10 equivalents of cholesteryl chloroformate (Alfa Aesar, USA) dissolved in DCM, together with 3 equivalents of triethyl amine to the peptides' resin. Conjugation of D-erythro-Dihydrosphingosine (D-erythro sphinganine), (Matreya, LCC, USA) to the N- or C-terminus of a peptide (compound 1) was performed as followed: first, 10 equivalents of N,N′-Disuccinimidyl carbonate (DSC) (Chem-Impex Internatinal, USA) and 20 equivalents of N,N-Diisopropylethylamine (DIEA) were added to the resin for 2 hours in dimethylformamide (DMF). Then, 2 equivalents of sphinganine and 2 equivalents of DIEA were added for overnight incubation in DMF anhydrous. The conjugation of D-erythro-Sphingosine (Matreya, LCC, USA) to the peptides (compound 5) was performed as described for compound 1. Addition of NBD-F, fluoride (4-fluoro-7-nitrobenzofurazan) (NBD) fluorescent probe (Biotium, USA) to the N- or C-terminus of selected peptides was performed in DMF for 1 hour. All peptides were cleaved from the resin by a TFA:DDW:TES (93.1:4.9:2 (v/v)) mixture, and purified by reverse-phase high-performance liquid chromatography (RP-HPLC) to >95% homogeneity. The molecular weight of the peptides was confirmed by platform LCA electrospray mass spectrometry. For hydrophobicity tests the peptides were eluted with a flow rate of 0.6 ml/min using a two-step linear gradient from CH3CN/H2O 20:80 v/v to CH3CN/H2O 95:5 v/v in 30 min and from CH3CN/H2O 95:5 v/v to CH3CN/H2O 95:5 in 10 min v/v on an analytical C2 column (7 μm particle size, pore size 100 Å) from Nucleosil.

HXB2 virus infectivity assay. Fully infectious HIV-1 HXB2 concentrated virus stock was a kind gift from the AIDS Vaccine Program, SAIC. The infectivity of HIV-1 HXB2 was determined using the TZM-bl cell line as a reporter. Cells were added (2×104 cells/well) to a 96-well clear-bottomed microtiter plate with 10% serum supplemented Dulbecco's modified eagle medium (DMEM). Plates were incubated at 37° C. for 18-24 hours to allow the cells to adhere. The media were then aspirated from each well and replaced with serum-free DMEM containing 40 micrograms/mL DEAE-dextran. Stock dilutions of each peptide were prepared in dimethyl sulfoxide (DMSO) so that each final concentration was achieved with 1% dilution. Upon addition of the peptides, the virus was added to the cells diluted in serum-free DMEM containing 40 micrograms/mL DEAE-dextran. The plate was then incubated at 37° C. for 18 hours to allow the infection to occur. Luciferase activity was analyzed using the Steady-Glo Luciferase assay kit (Promega, Madison Wis.). Sometimes washing experiments were performed as follows: peptides were pre-incubated with TZM-bl cells at 37° C. for 1 hour, followed by three washes with culture medium to remove unbound peptides and the addition of HXB2 viruses to start the infection. After 18 hours, the antiviral activities of the remaining peptides that survive the washing were determined by measuring Luciferase activity.

Fitting of the data points was performed according to the following equation, derived from Hills' equation as previously described (Wexler-Cohen et al. Faseb J 24, 4196-4202, 2010):

$$Y ( x ) = Bx ( \frac{{A}^{C}}{{X}^{C} + {A}^{C}} ) \qquad ( 1 )$$

In brief, in this equation B is the maximum value; therefore, it equals 100% fusion, A is the value of an inhibitory concentration at 50% viral infectivity (IC50), and c represents Hill's coefficient. For the fitting, we uploaded the X and Y values of the data into a nonlinear least squares regression (curve fitter) program that provided the IC50 value (parameter A).

Infection with pseudotyped viruses. 293T cells were cotransfected with the pNLLuc plasmid and LAI ENV, AD8 ENV, or VSV-G ENV plasmids. After 48 hours of transfection the viral supernatant was harvested and titrated with the TZM-bl cells. The pseudoviruses thus generated have the same core and differ only by the fusion protein present on their surface. These pseudoviruses are only able to undergo a single round of infection with their target cells. The target cells were resuspended in medium without serum containing 40 micrograms/mL DEAE-dextran and incubated in a 96-well plate. Stock dilutions of each peptide were prepared in DMSO so that each final concentration was achieved with 1% dilution. Upon addition of the peptides, the pseudovirus was added to the cells and then diluted in serum-free RPMI containing 40 micrograms/mL DEAE-dextran. The plate was then incubated at 37° C. for 2 days to allow the infection to occur. Luciferase activity was analyzed using the Steady-Glo Luciferase assay kit. To assess viral fusion with Jurkat, clone E6-1 T-cells, LAI pseudotyped viruses were used. The Jurkat cells were used at 1.5×10⁴ cells/well and the infection was carried out for 3 days.

Fuzeon-resistant virus infection. Virus stocks of the wild-type LAI and V38E mutant were prepared with the use of molecular clones. Briefly, 293T cells were transfected with infectious molecular clones. The virus supernatant was collected 48 hours post transfection, cleared of cellular debris by centrifugation, aliquoted, and stored at −70° C. The wild-type virus contains an NL4-3 backbone with an LAI ENV. For the mutant, a V38E mutation has been generated in the LAI ENV by site-directed mutagenesis as described earlier. Infection with those viruses was performed as previously described for the HXB2 virus.

Cell-cell fusion assay. Effector cells were the ENV expressing cells HL2-3, a Hela derived cell-line which constitutively expresses the HXB2 strain of the HIV-1 ENV glycoprotein along the Tat protein, and as target cells, TZM-bl cells were used. The fusion of HL2-3 cells with TZM-bl cells was assessed through luciferase expression. The TZM-bl cells were seeded at 2×10⁴ cells/well overnight in a 96 well plates. The media was then aspirated from each well and replaced with serum-free DMEM containing 40 micrograms/mL DEAE-dextran. Stock dilutions of each peptide were prepared in DMSO so that each final concentration was achieved with 1% dilution. Upon addition of the peptides, the HL2-3 cells were added to the TZM-bl cells in serum-free DMEM containing 40 micrograms/mL DEAE-dextran at a 1 to 1 cell ratio. The cells were cocultured at 37° C. for 6 hours to allow the fusion to occur. Luciferase activity was analyzed using the Steady-Glo Luciferase assay kit.

Lipid vesicle preparation. Large unilamellar vesicles (LUV) were prepared from egg phosphatidylcholine (PC), cholesterol (Chol) and egg yolk sphingomyelin (SM) (Sigma Chemical Co., Israel). A dried film of lipids containing a total of 2 mg of PC:SM:Chol (1:1:1) or 2 mg of PC:Chol (9:1) was suspended in PBS and vortexed for 1.5 min. The lipid suspension underwent five cycles of freezing-thawing and then extrusion through polycarbonate membranes with 1 μm and 0.1 μm diameter pores for 25 times.

Peptide binding to LUV. The degree of peptide association with lipid vesicles was measured by adding lipid vesicles to 0.1 μM fluorescently NBD-labeled peptides at room temperature. The fluorescence intensity was measured as a function of the lipid:peptide molar ratio, with excitation set at 467 nm (10-nm slit) and emission set at 530 nm (10-nm slit). To determine the extent of the lipid's contribution to any given signal, the readings after the addition of lipid vesicles were subtracted as background from the recorded fluorescence intensity. The affinity constants were then determined by steady state affinity model using non-linear least-squares (NLLSQ). The NLLSQ fitting was done using the following equation:

$$Y ( x ) = \frac{{K}_{a} \times X \times {F}_{max}}{1 + {K}_{a} \times X} \qquad ( 2 )$$

where X is the lipid concentration, Y(x) is the fluorescence emission, Fmax is the maximal difference in the emission of NBD-labeled peptide before and after the addition of lipids (it represents the maximum peptide bound to lipid), and Ka is the affinity constant.

Secondary structure determination. Circular Dichroism (CD) measurements were performed by using an AppliedPhoto physics spectropolarimeter. The spectra were scanned using a thermostatic quartz cuvette with a path length of 1 mm. Wavelength scans were performed at 25° C.; the average recording time was 15 sec, in 1-nm steps, in the wavelength range of 190-260 nm. Each peptide concentration was 10 μM in HEPES buffer (5 mM, pH 7.4).

Statistical analysis. Data representing the means±standard deviation (s.d.) of the results from several experiments were compared by one-tailed Student's t-test.

### Example 1

### Sphinganine Exclusively Endows Antiviral Activity to a Non-Active Short Conserved gp41 N-Peptide

The 17-mer sequence, termed as the pocket, is a conserved domain within the HIV-1 gp41 protein core. This is a deep cavity on the surface of the grooves of the NHR trimer that is important for stabilizing the trimer; it interacts with the CHR to maintain the stability of the gp41 core. However, a synthetic N-peptide derived from the pocket domain (termed N17 having the amino acid sequence of SEQ ID NO:1) was not active in inhibiting viral infectivity up to 4 μM, the maximal concentration tested (Table 1).

TABLE 1
Antiviral activity and biochemical properties of N17 peptides and their
different lipid conjugates

| | | Hydrophobicity | Virus-cell fusion |
|---|---|---|---|
| | Helicity (%) | (min) | IC₅₀ (μM) |
| N17 (SEQ ID NO: 1) | 20 | 18.2 | >4 |
| Palmitic acid-N17 | <10 | 26.7 | >4 |
| Cholesterol-N17 | <10 | 28.9 | >4 |
| Tocopherol-N17 | <10 | 28.9 | >4 |
| Sphinganine-N17 | <10 | 27.2 | 0.121 ± 0.036 |

Virus-cell fusion IC₅₀ (μM): TZM-bl cells were infected with fully contagious HIV in the presence of different compounds. The IC₅₀ for each compound was calculated as described in the section of Methods. Results are mean±standard deviation (s.d), n=3.

Hydrophobicity (min): Hydrophobicity was analyzed by RP-HPLC. The retention time of each compound is presented.

Helicity (%): The percentage of α-helical structure was determined as described in the section of Materials and Methods.

We conjugated to N17 the following hydrophobic moieties (FIG. 1B, C): (i) dihydrosphingosine (sphinganine), which forms the backbone of dihydrosphingomyelin, (ii) cholesterol, and (iii) palmitic acid, which is the building block in many lipids including phospholipids. For a comparison, we also conjugated tocopherol, a hydrophobic non-cellular compound, to N17. The hybrid compounds were tested for their ability to inhibit viral infectivity. Notably, the inhibitory activity of N17 was dependent on the nature of the lipid moiety (Table 1, FIG. 2). Sphinganine was the most prominent one and potentiated the antiviral activity of N17 into the nanomolar concentration range. This exclusive inhibitory activity of sphinganine-based peptides (sphingopeptides) was further observed in a wider spectrum of viral entry systems consisting of different HIV strains (wild-type HXB2 and LAI) and different target cells (TZM-bl reporter cells and Jurkat T cells), as well as in cell-cell fusion assays (Table 1, FIG. 2).

Increased antiviral activity of short peptides is sometimes attributed to increased hydrophobicity or increased α-helical content of the compound via conjugation to a specific moiety. Therefore, we checked the apparent hydrophobicity of N17 and its lipid conjugates (Table 1). The most hydrophobic compounds were the tocopherol and cholesterol conjugates. Sphinganine and palmitic acid conjugates exhibited similar medium hydrophobicity and N17 alone was the least hydrophobic. Clearly, there was no correlation between the extent of hydrophobicity and antiviral activity in this case. In addition, there was no increase in the α-helical content of N17 upon conjugation (Table 1).

### Example 2

### The Specificity of Anti-HIV Activity of Sphingopeptides is Determined by Both the Peptides and Their Lipid Moiety

We further investigated the interplay between sphinganine and its conjugated peptide in inhibiting viral infectivity. Viruses were constructed to have the same HIV core with three different ENV: LAI (CXCR4 tropic), AD8 (CCR5 tropic), and vesicular stomatitis virus G (VSV-G). The three constructed viruses were allowed to infect TZM-bl cells in the presence of increasing concentrations of sphingopeptides (FIG. 3A). Antiviral activity was observed for LAI and AD8 but not for VSV-G. Moreover, sphingosine, the building block of many sphingolipids and an analogue of sphinganine, was conjugated to N17 peptides (FIG. 3B). Sphingopeptides that contained the sphingosine backbone also showed potent antiviral activity (FIG. 3C).

The importance of peptide interactions to the overall activity of the molecules was examined using sequence mutagenesis. Mutated sphingopeptides were prepared by replacing N17 peptides residues in positions a and d of the helical wheel, termed N17m(a,d), thus knocking out their ability to self-assemble (FIG. 3D, E). Alternatively, we randomly reorganized N17 sequence to give scrambled peptides (FIG. 3E). None of the mutant N17 peptides or their sphinganine conjugates showed antiviral activity (FIG. 3F). Additionally, a 17-mer N-peptide was synthesized from the same region as N17 but shifted in its sequence from the gp41 pocket (the peptide termed ShN17 having the amino acid sequence of SEQ ID NO:2, FIG. 4A). Sphingopeptides comprising the ShN17 sequence exhibited antiviral activity that was 7.3-fold lower than the ones with the pocket sequence (Table 2, FIG. 4B). The addition of sphinganine alone, up to 1 μM (a concentration at which sphinganine-N17 completely blocks fusion), did not affect viral infectivity (FIG. 4B).

### Example 3

### Sphinganine Potentiates the Activity of Short N- and C-Peptides in Wild-Type and Fuzeon-Resistant Virus

Changing the orientation of sphinganine towards N17 was achieved by adding lysine to its C-terminus, enabling C-terminal lipid conjugation. The addition of the lysine did not alter the antiviral activity of the compound according to its inhibitory concentration at 50% infectivity (IC₅₀) of 121 nM±36 nM and 116 nM±12 nM to sphinganine-N17 and sphinganine-N17K, respectively (Table 2, FIG. 4B and FIG. 4C). The inhibitory potentiation of sphinganine was still powerful via C-terminal conjugation, exhibiting an IC₅₀ value of 287 nM±143 nM to N17K-sphinganine (FIG. 4C). We then investigated whether the ability of sphinganine to potentiate the activity of short N-peptides could be exploited to other peptides from the gp41 core. Therefore, a short 19-mer C-peptide from the CHR, termed DP19 (SEQ ID NO:3) was synthesized (FIG. 1B, FIG. 4A). DP19K-sphinganine was remarkably more potent than DP19K; exhibiting an IC₅₀ value of 350 nM±60 nM (Table 2, FIG. 4D). Accordingly, this amplification of fusion inhibition is not restricted only to N-peptides. It can be implemented in other short fragments from different regions within the gp41 core.

The GIV sequence within the NHR of gp41 (FIG. 4A) is well established as a site for escape mutations of the virus for the drug Fuzeon, which leads to viral resistance. We introduced to the HIV construct a mutation in the GIV sequence (V38E) that considerably weakened the antiviral activity of Fuzeon, whereas sphinganine-N17 preserved its potent antiviral activity (Table 2, FIG. 4E). The results further showed that truncated peptides of even 10 amino acids (e.g., SEQ ID NO: 11) as well as incorporation of D-amino acids (SEQ ID NO:12) retained their antiviral activity (Table 3).

TABLE 2
Designation and antiviral activity of the HIV peptides and their lipid
conjugates in a fully infectious HIV-1 system.

| Name | X | Sequence | Z | IC₅₀ (nM) |
|---|---|---|---|---|
| N17 | H | x-LLQLTVWGIKQLQARIL | | >4000 |
| C16-N17 | Palmitic acid | | | >4000 |
| Chol-N17 | Cholesterol | | | >4000 |
| Tocopherol-N17 | Tocopherol | | | >4000 |
| Sphing-N17 | Sphinganine | | | 121 ± 36 |
| Sphingosine-N17 | Sphingosine | | | 208 ± 5 |
| N17K | H | x-LLQLTVWGIKQLQARILK (δ-NHz) | H | >4000 |
| Sphing-N17K | Sphinganine | | H | 118 ± 18 |
| N17K-sphing | H | | Sphinganine | 291 ± 102 |
| N17 scrambled | H | x-QKIARLQTLWLQIGVLL | | >4000 |
| Sphing-N17 scrambled | Sphinganine | | | >4000 |
| N17mut (a, d) | H | x-LEQLSVWGSKQNQARRL | | >4000 |
| Sphing-N17mut (a, d) | Sphinganine | | | >4000 |
| N17mut (e, g) K | H | x-ELQLTQWKIKQLLARILK (δ-NHz) | H | >4000 |
| Sphing-N17mut | Sphinganine | | H | 120 ± 30 |
| (e, g) K | | | | |
| N17mut (e, g) | H | | Sphinganine | 120 ± 40 |
| K-sphing | | | | |
| Sphing-ShN17 | Sphinganine | x-LQARILAVERYLKDQQL | | 886 ± 102 |
| DP19K | H | x-YTSLIHSLIEESQNQQEKNK (δ-NHz) | H | 2213 ± 507 |
| DP19K-sphing | H | | Sphinganine | 350 ± 60 |
| N14 | H | x-LLQLTVWGIKQLQA | | >4000 |
| Sphing-N14 | Sphinganine | | | 500 ± 100 |

TABLE 3
Antiviral activity of exemplary short
sphingopeptides of the invention in
fully infectious HIV-1 system and
in cell-cell fusion system.

| Desig- | | | |
|---|---|---|---|
| nation | Sequence/formula | IC₅₀ (nM)^{a} | IC₉₀ (nM)^{b} |
| PBDK | TTWMEWDREINNYTK | 5348 ± 1700 | >10000 |
| PBDK- | TTWMEWDREINNYTK- | 467 ± 140 | 1089 ± 100 |
| Sphing | sphing | | |
| Scrambled- | TTEIWREDTNYMWNK- | 895 ± 70 | 6263 ± 3670 |
| Sphing | sphing | | |
| Truncated- | TWMEWDREIK- | 1331 ± 270 | 2483 ± 320 |
| Sphing | sphing | | |
| D-peptide- | IWMEWDREIK- | 288 ± 156 | 2668 ± 540 |
| sphing^{c, d} | sphing | | |

^{a}Inhibitory concentration of the compound in 50% virus-cell fusion.
^{b}Inhibitory concentration of the compound in 90% virus-cell fusion.
^{c}In bold are D-amino acids incorporations.
^{d}The inhibitory concentrations of the D-peptides were obtained from a cell-cell fusion system mediated by the HIV-1 envelope (TZM-bl target cells and HL 2/3 effector cells).

### Example 4

### Sphingopeptides Strongly Binds and Accumulates in the Cell Membrane

The ability of sphingopeptides to bind and accumulate in the cell membrane was examined by pre-incubating sphinganine-N17 with target cells, followed by washing before adding the virus to initiate infection. We calculated the IC₅₀ of the peptide with or without washing the cells (IC₅₀ 1 or IC₅₀ 2, respectively) to evaluate the ability of residual peptides to sustain inhibitory potency (FIG. 5A). Surprisingly, the IC₅₀ value of sphinganin-N17 increased only by 5-fold following cell washing. We compared this to gp41 peptide fusion inhibitors with different membrane-binding properties: C34 (depicted in FIG. 4A), which poorly binds membranes and Fuzeon, which has an intrinsic membrane-binding ability The IC₅₀ of C34 dramatically increased by 305-fold, whereas a 36-fold increase was observed for the IC50 of Fuzeon following cell washing.

We investigated whether sphinganine-N17 could also inhibit when bound to the viral membrane by adding the compound to target cells before adding the virus or immediately after adding it. In the second procedure, both viral and cell membranes are presented at the time of addition and the sphingopeptides could theoretically bind to any of them. If binding to the viral membrane has a more pronounced effect, we might expect differences in the fusion inhibition curves of the compound, depending on the time that the virus was added. Importantly, no difference between the viral infectivity curves was observed for sphing-N17 (FIG. 5B).

### Example 5

### Sphingopeptides Preferably Bind Lipid Vesicles Enriched in Sphingomyelin and Cholesterol

The degree of compound association with lipid vesicles was estimated by NBD-labeled compound titration with large unilamellar vesicles (LUV). The membrane-binding affinity constants (Ka), derived from the binding equation (equation 2 in Materials and Methods), are presented in Table 4 for sphing-N17, C16-N17, and Fuzeon associated with vesicles enriched in sphingomyelin and cholesterol, and with vesicles comprising only phosphatidylcholine and cholesterol. Palmitic acid was chosen for the binding assay since it exhibited hydrophobicity close to sphinganine, but with no antiviral activity (Table 1). Sphing-N17 and C16-N17 had higher binding affinities to both types of lipids, compared with Fuzeon. Interestingly, sphing-N17 preferably binds lipid vesicles enriched in sphingomyelin and cholesterol (Table 4). Fuzeon also exhibited some preference toward these vesicles but to a lesser extent, and C16-N17 retained binding affinity similar to both types of vesicles.

TABLE 4
Sphinganine-N17 preferably binds lipid vesicles enriched in
sphingomyelin and cholesterol.

Membrane-binding constant (M⁻¹)

| | PC:Chol (9:1) | PC:SM:Chol (1:1:1) |
|---|---|---|
| Sphing-N17 | 4.7 × 10⁵ ± 1 × 10⁵ | 8.4 × 10⁴ ± 1.4 × 10⁴ |
| C16-N17 | 1.7 × 10⁵ ± 0.3 × 10⁵ | 1.5 × 10⁵ ± 0.7 × 10⁵ |
| Fuzeon | 6.4 × 10⁴ ± 1.4 × 10⁴ | 3.1 × 10⁴ ± 0.5 × 10⁴ |

Compound association with lipid vesicles was estimated by NBD-labeled compound titration with large unilamellar vesicles. The mole ratio between phosphatidylcholine (PC), sphingomyelin (SM), and cholesterol (Chol) is presented for each vesicle. The membrane-binding constants (Ka) were calculated from equation 2 in the Methods. Results represent the mean±s.d. of the fitting curve, n=3.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

1. A sphingolipid-peptide conjugate comprising an isolated peptide of up to 22 amino acid residues coupled to a sphingolipid moiety, the peptide comprising the amino acid sequence selected from the group consisting of:
LLQLTVWGIKQLQARIL (SEQ ID NO:1);
LQARILAVERYLKDQQL (SEQ ID NO:2);
YTSLIHSLIEESQNQQEKN (SEQ ID NO:3); and
TTWMEWDREINNYT (SEQ ID NO: 4);
or an analog, a derivative or fragment thereof.

2. The sphingolipid-peptide conjugate according to claim 1, wherein the sphingolipid moiety is conjugated to the N-terminus or to the C-terminus of the isolated peptide.

3. (canceled)

4. The sphingolipid-peptide conjugate according to claim 1, wherein the sphingolipid moiety is selected from sphinganine and sphingosine.

5. The sphingolipid-peptide conjugate according to claim 1, wherein the peptide further comprises 1-4 basic amino acid residues contiguous to at least one of the peptide's termini.

6. The sphingolipid-peptide conjugate according to claim 5, wherein the basic amino acid residue is selected from Lysine and Arginine residues.

7. The sphingolipid-peptide conjugate according to claim 1, wherein the isolated peptide comprises or consists of the amino acid sequence as set forth in LLQLTVWGIKQLQARIL (SEQ ID NO:1).

8. (canceled)

9. The sphingolipid-peptide conjugate according to claim 6, wherein the isolated peptide comprises the amino acid sequence as set forth in LLQLTVWGIKQLQARILK (SEQ ID NO:5).

10. The sphingolipid-peptide conjugate according to claim 1, wherein the analog or fragment of SEQ ID NO:1 has the amino acid sequence selected from the group consisting of: ELQLTQWKIKQLLARIL (SEQ ID NO:6);
ELQLTQWKIKQLLARILK (SEQ ID NO:7); and
LLQLTVWGIKQLQA (SEQ ID NO:8).

11. The sphingolipid-peptide conjugate according to claim 1, wherein the isolated peptide comprises or consists of the amino acid sequence as set forth in LQARILAVERYLKDQQL (SEQ ID NO:2).

12. (canceled)

13. The sphingolipid-peptide conjugate according to claim 1, wherein the isolated peptide comprises or consists of the amino acid sequence as set forth in YTSLIHSLIEESQNQQEKN (SEQ ID NO:3).

14. (canceled)

15. (canceled)

16. The sphingolipid-peptide conjugate according to claim 6, wherein the isolated peptide comprises or consists of the amino acid sequence as set forth in YTSLIHSLIEESQNQQEKNK (SEQ ID NO:9).

17. (canceled)

18. The sphingolipid-peptide conjugate according to claim 1, wherein the isolated peptide comprises or consists of the amino acid sequence as set forth in TTWMEWDREINNYT (SEQ ID NO:4).

19. The sphingolipid-peptide conjugate according to claim 1, wherein the fragment of SEQ ID NO:4 consists of the amino acid sequence as set forth in TWMEWDREIK (SEQ ID NO:11).

20. The sphingolipid-peptide conjugate according to claim 1, wherein the isolated peptide comprises the amino acid sequence as set forth in TTWMEWDREINNYTK (SEQ ID NO:10).

21. (canceled)

22. The sphingolipid-peptide conjugate according to claim 1, comprising at least one D amino acid.

23. (canceled)

24. The sphingolipid-peptide conjugate according to claim 22, wherein the peptide comprises the amino acid sequence as set forth in IwMewDREIK (SEQ ID NO: 12), wherein “w” denotes D-Trp and “e” denotes D-Glu.

25. A pharmaceutical composition comprising as an active ingredient a sphingolipid-peptide conjugate according to claim 1 and a pharmaceutically acceptable carrier or diluent.

26-29. (canceled)
